(19) United States
(12) Patent Application Publication
(10) Pub. No.: US 2012/0039917 A1
(43) Pub. Date: Feb. 16, 2012

(54) IMMUNOMODULATING ACTIVITIES

(76) Inventors: Alan James Husband, Pyrmont (AU); David Brown, North Ryde (AU); Patries Herst, Wellington (NZ)

(21) Appl. No.: 13/061,112

(22) PCT Filed: Aug. 28, 2009
(86) PCT No.: PCT/AU2009/001117

§ 371 Date: Nov. 1, 2011

(30) Foreign Application Priority Data
Aug. 29, 2008 (AU) 2008904483

(51) Int. Cl. A61K 39/00; A61P 37/02; C12N 5/02

(52) U.S. Cl. 424/184.1; 435/375

(57) ABSTRACT

Composition of isoflavonoids and chromanols and use of them as immunomodulators and as inhibitors of T-cell or T-lymphocyte proliferation. Treatment of disorders involving abnormal proliferation or activity of T cells. Formula (I) where A is hydrogen or optionally substituted phenyl and R1 represents hydroxy, alkoxy, halo or an ester and R2-R8 represent hydogen, hydroxy, alkyl etc.

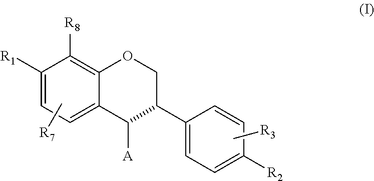

### FIELD OF THE INVENTION

The present invention relates generally to methods and compositions utilised to modulate the immune system. In particular the invention relates to the use of isoflavonoid compounds to modulate the activity and/or proliferation of lymphocytes.

### BACKGROUND OF THE INVENTION

Phenoxodiol (2H-1-benzopyran-7-0, 3-(hydroxylphenyl); isoflav-3-en-4′,7-diol; PXD) is a synthetic analogue of the plant isoflavone genistein. In vitro studies using various cancer cell lines and in vivo experiments in animal models have demonstrated the ability of PXD to act as an anticancer agent and as a chemosensitizer in conjunction with various chemotherapeutic agents (such as carboplatin, gemcitabine and topotecan) (see, for example, Alvero et al., 2007; Alvero et al., 2008). Based on such findings PXD was granted a fast track approval by the US Food and Drug Administration in 2004 and entered clinical trials in humans. The results from two different phase I clinical trials in 2006, where PXD was administered by intravenous infusion in late stage solid cancer patients, showed that the drug is well tolerated up to the dose of 30 mg/kg with minor side effects (Choueiri et al, 2006; de Souza et al, 2006). In both studies, some patients experienced stabilization of their disease up to 6 months after treatment. PXD has subsequently entered phase II and phase III clinical trials for the treatment of, hormone related cancers, including ovarian, cervical, prostate and breast cancer.

T cell activation and proliferation are normal and essential processes in the development of an immune response. However abnormal or dysregulated T cell activation and/or proliferation has been implicated in a number of pathological conditions, such as inflammatory disorders and autoimmune diseases. There remains a need for the development of new therapeutic approaches to modulate T cell activation or proliferation and the activity of proliferating T cells.

The present invention is predicated on the inventors' surprising findings that the potent chemotherapeutic molecule PXD is able to regulate specific immune functions, including the inhibition of rapidly proliferating T cells. These findings open up a range of hitherto unknown and unexpected therapeutic targets for, and therapeutic opportunities using, PXD and related compounds.

### SUMMARY OF THE INVENTION

According to a first aspect of the present invention there is provided use of a compound of formula I

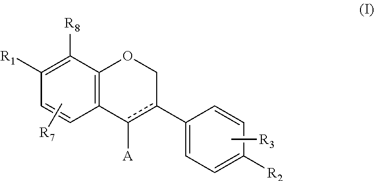

wherein
R₁ is hydroxy, alkoxy, halo or OC(O)R₉,
R₂ and R₃ are independently hydrogen, hydroxy, alkoxy, alkyl, halo or OC(O)R₉,
A is hydrogen or optionally substituted phenyl of the formula

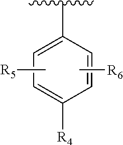

R₄, R₅ and R₆ are independently hydrogen, hydroxy, alkoxy, alkyl, amino, alkylamino, dialkylamino or OC(O)R₉,
R₇ and R₈ are independently hydrogen, hydroxy, alkyl, alkoxy or halo,
R₉ is hydrogen, alkyl, aryl, arylalkyl or amino, and the drawing “ [symbol] ” represents a single bond or a double bond,
   or a pharmaceutically acceptable salt or prodrug thereof,
   as an immunomodulatory agent.

In an embodiment the compound is selected from isoflav-3-en-4′,7-diol, 3-(4-hydroxyphenyl)-4-(4-methoxyphenyl)chroman-7-ol and 3-(4-hydroxyphenyl)-4-(4-methoxyphenyl)-8-methylchroman-7-ol. In a particular embodiment the compound is isoflav-3-en-4′,7-diol.

The compound may be administered as an immunomodulatory agent, in an immunomodulating effective amount, to a mammal in need thereof. The compound may be administered in the form of a pharmaceutical composition.

In a particular embodiment the immunomodulatory activity of the compound of formula I comprises the inhibition of the proliferation and/or activity of proliferating T cells. Typically the T cells are abnormally or rapidly proliferating T cells. Alternatively the T cells may be responder T cells. The compound may induce or promote apoptosis of proliferating T cells, typically rapidly or abnormally proliferating T cells. The inhibition of activity may comprise inhibiting plasma membrane electron transport in proliferating T cells, typically rapidly or abnormally proliferating T cells. Accordingly, the compound may be administered as an immunomodulatory agent, in an immunomodulating effective amount, to a mammal in need thereof. The mammal may be suffering from, or susceptible to, a disease or condition associated with abnormal proliferation or stimulation of T cells.

According to a second aspect of the invention there is provided a method for inhibiting the activity and/or proliferation of proliferating T cells, the method comprising exposing the proliferating T cells to an effective amount of at least one compound of formula I as described herein or a pharmaceutically acceptable salt or prodrug thereof.

The exposure of the proliferating T cells to the at least one compound may occur in vivo or ex vivo.

The proliferating T cells may reside in, or be derived from, a subject suffering from or predisposed to a disease or condition associated with abnormal proliferation or stimulation of T cells.

According to a third aspect of the invention there is provided a method of modulating the immune system in a mammal, the method comprising administering to the mammal an immunomodulating effective amount of at least one compound of formula I as described herein or a pharmaceutically acceptable salt or prodrug thereof.

In an embodiment the compound may inhibit the activity and/or proliferation of proliferating T cells, typically abnormally or rapidly proliferating T cells.

According to a fourth aspect of the invention there is provided a method for the treatment or prevention of a disease or condition associated with abnormal proliferation or stimulation of T cells, the method comprising administering to a mammal in need thereof an immunomodulating effective amount of a compound of formula I as described herein or a pharmaceutically acceptable salt or prodrug thereof.

According to a fifth aspect of the invention there is provided the use of a compound of formula I as described herein, or a pharmaceutically acceptable salt or prodrug thereof, for the manufacture of a medicament for the treatment or prevention of a disease or condition associated with abnormal proliferation or stimulation of T cells.

According to an sixth aspect of the invention there is provided a method for augmenting a treatment regime for a subject suffering from a disease or condition associated with the abnormal proliferation or stimulation of T cells, the method comprising administering to the subject an immunomodulating effective amount of a compound of formula I as described herein or a pharmaceutically acceptable salt or prodrug thereof.

Typically in accordance with the above aspects and embodiments the subject is human. In other embodiments, the subject may be a mammal selected from the group consisting of, but not limited to: primate, ovine, bovine, canine, feline, porcine, equine and murine.

### BRIEF DESCRIPTION OF THE DRAWINGS

The present invention will now be described, by way of non-limiting example only, with reference to the accompanying drawings.

FIG. 1. PXD inhibits PMET, proliferation and viability of proliferating T cells. (A). PMET was measured as WST-1/PMS reduction in the presence of different concentrations of PXD in proliferating (closed circles) and resting T cells (open circles). (B) Proliferation was measured by MTT reduction in the presence of different concentrations of PXD in proliferating (closed circles) and resting T cells (open circles). (C) Viability was measured as Trypan blue exclusion of proliferating T cells (closed triangles) exposed to 10 μM PXD or controls (closed circles) and of resting T cells (open triangles) exposed to 10 μM PXD or controls (open circles). Controls were DMSO at the same concentration as in PXD treatment. Inhibition of all parameters was observed in proliferating T cells but not in resting T cells. Results are presented as the average±SEM of three separate experiments.

FIG. 2. PXD increases the extent of apoptosis of proliferating T cells. (A) to (D): resting T cells; (E) to (H): proliferating T cells. NE and C/G are scatter plots of T cells treated with 0.1% DMSO (control) and 10 μM PXD for 24 h respectively. The extent of apoptosis was measured by the percentage AV⁺/PI⁺ cells from the gated CD3⁺ T cell populations in the corresponding scatter plots. B/F and D/H are AV/PI plots of control and PXD-treated T cells respectively. PXD increased the percentage of AV⁺/PI⁺ cells in proliferating but not in resting T cells. Results are representative of three separate experiments.

FIG. 3. Exposure to PXD eliminates proliferating responder T cells in HLA-mismatched MLRs.

Responder PBMC cells were exposed to HLA-mismatched γ-irradiated stimulator cells in the presence of 0.1% DMSO (A and B) and 10 μM PXD (C and D) on day 0 and analysed on day 8. A and C are scatter plots of control and PXD-treated responder PBMC respectively. Gates indicate the CD3⁺ T lymphocyte populations. B and D are proliferation plots of the corresponding CD3⁺ T lymphocyte population, showing viable resting populations (CSFE^{hi}AV⁻) in both control and PXD-treated MLRs and viable proliferating populations (CFS^{lo}AV⁻) in the control MLR only. Results are representative of three separate experiments.

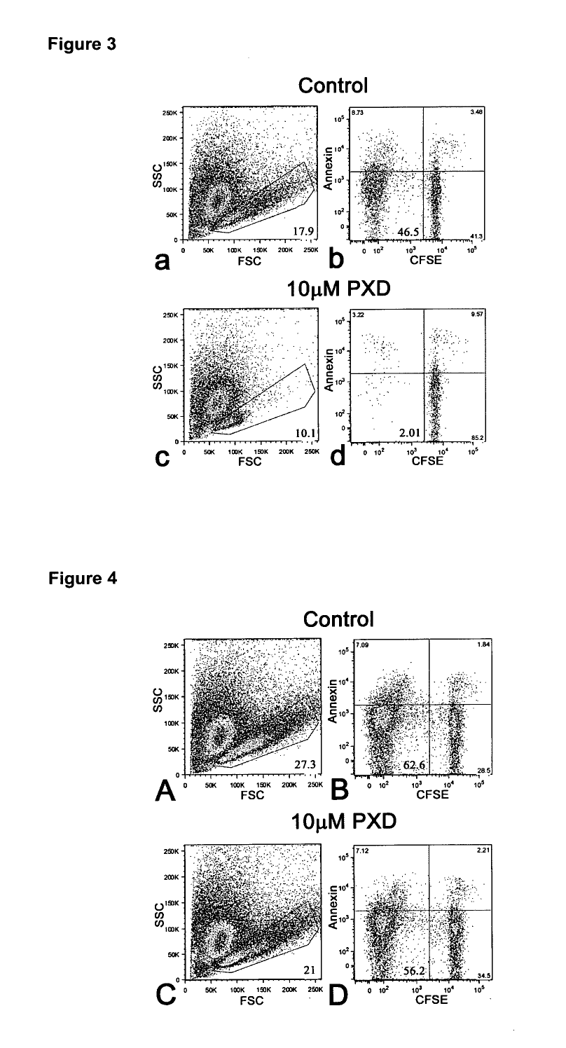

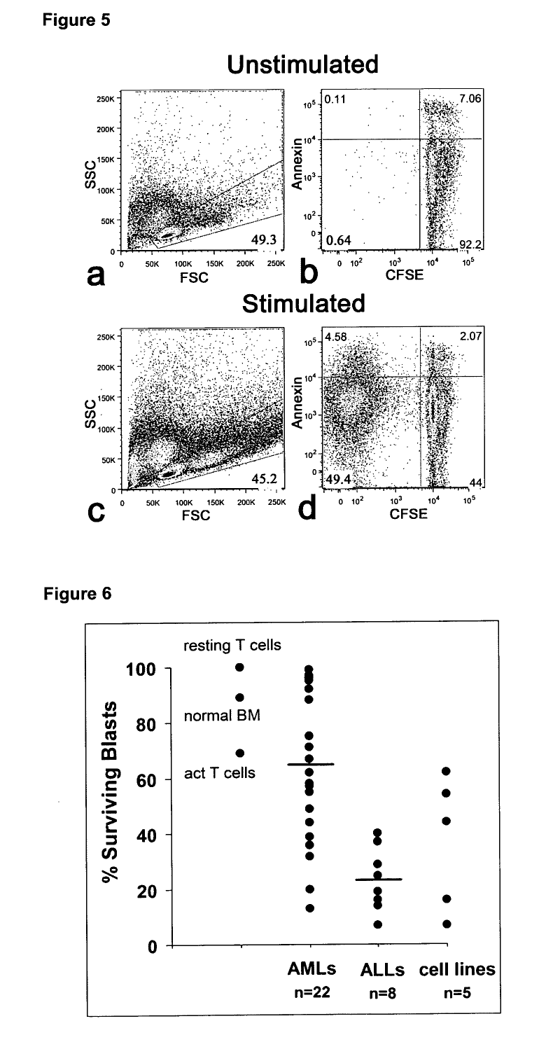

FIG. 4. Transient exposure of unstimulated T cells to PXD does not affect their ability to respond in HLA-mismatched MLR. Responder cells were pre-incubated with 0.1% DMSO (control) or 10 μM PXD for 24 h, washed twice in fresh medium, mixed with HLA-mismatched γ-irradiated stimulator cells on day 0 and analysed by FACS on day 8. See FIG. 3 for an explanation of plots. Results are representative of two separate experiments.

FIG. 5. Transient exposure of resting responder T cells to PXD does not affect their ability to respond in a subsequent HLA-mismatched third party MLR. Viable resting responder T cells (CD3⁺CSFE^{hi}AV⁻) were sorted on day 8 of an HLA-mismatched MLR and either incubated for a further 8 days (A and B) or restimulated in a second subsequent third party MLR, with HLA-mismatched γ-irradiated stimulator cells from a third person (C and D). Only restimulated responder T cells showed strong proliferation in the subsequent MLR (CSFE^{lo} population in D but not in B. Results are representative of two separate experiments.

FIG. 6. Sensitivity of different cell types to PXD. Different cell types were incubated with 10 μM PXD for 24 h and viability was determined by AV/PI staining and reported as [% viable blasts after PXD exposure]/[% viable blasts after 0.1% DMSO exposure]. Percentage values are as follows: AML-derived HL60: 16±5 and HL60p°: 54±6, ALL-derived MOLT-4: 44±4, MM-derived U226: 59±6 and RPMI 8226: 12±4, primary AML blasts: 64±5, primary ALL blasts: 23±4, normal BM: 89±5, proliferating T cells: 69±4, resting T cells: 98±6. Results from cell lines are presented as the average±SEM of at least 3 separate experiments, results from bone marrow samples are presented as the average±SD of single experiments performed in duplicate.

### DETAILED DESCRIPTION OF THE INVENTION

Throughout this specification and the claims which follow, unless the context requires otherwise, the word “comprise”, and variations such as “comprises” or “comprising”, will be understood to imply the inclusion of a stated integer or step or group of integers or steps but not the exclusion of any other integer or step or group of integers or steps.

The articles “a” and “an” are used herein to refer to one or to more than one (i.e., to at least one) of the grammatical object of the article. By way of example, “an element” means one element or more than one element.

As used herein the terms “treating”, “treatment”, “preventing” and “prevention” refer to any and all uses which remedy a condition or symptoms, prevent the establishment of a condition or disease, or otherwise prevent, hinder, retard, or reverse the progression of a condition or disease or other undesirable symptoms in any way whatsoever. Thus the terms “treating” and “preventing” and the like are to be considered in their broadest context. For example, treatment does not necessarily imply that a patient is treated until total recovery. Rather, “treatment” encompasses reducing the severity of, or delaying the onset of, a particular disorder. In the context of some disorders, methods of the present invention involve “treating” the disorder in terms of reducing or ameliorating the occurrence of a highly undesirable event associated with the disorder or an irreversible outcome of the progression of the disorder but may not of itself prevent the initial occurrence of the event or outcome. Accordingly, treatment includes amelioration of the symptoms of a particular disorder or preventing or otherwise reducing the risk of developing a particular disorder.

As used herein the term “effective amount” means an amount or dose of a compound or pharmaceutical composition comprising such a compound, which includes within its meaning a non-toxic but sufficient amount or dose of a compound or pharmaceutical composition so as to provide the desired effect. The exact amount or dose required will vary from subject to subject depending on factors such as the species being treated, the age and general condition of the subject, the severity of the condition being treated, the particular agent being administered and the mode of administration and so forth. Thus, it is not possible to specify an exact “effective amount” or “effective dose”. However, for any given case, an appropriate “effective amount” or “effective dose” may be determined by one of ordinary skill in the art using only routine experimentation.

The term “immunomodulating” as used herein with reference to the “immunomodulating effective amount” of a compound means an amount or dose of the compound that is sufficient to modulate the immune system or immune response as desired. The immunomodulating amount may be consistent with or different from a “therapeutic amount” of the compound, where a therapeutic amount is an amount that is sufficient to have a non-immunomodulatory, therapeutic effect against a particular disease or condition. For example, an immunomodulatory amount may be a subtherapeutic amount, that is, a smaller amount or dose of the compound administered to achieve an immunomodulatory effect than the amount or dose required to be administered to achieve a non-immunomodulatory therapeutic effect.

As used herein the term “inhibit” means to retard, prevent, decrease or reduce. Thus, in the context of the present invention, the ability of a compound described herein to “inhibit” the proliferation of a cell means that the compound may reduce proliferation, inhibit or prevent continued cell proliferation, or retard or prevent the initiation of proliferation. Those skilled in the art will appreciate that also encompassed by the inhibition of cell proliferation, in its broadest sense, is the induction or promotion of cell death, typically apoptosis. Inhibition of proliferation or activity encompasses total or partial inhibition; the degree of inhibition is sufficient to reduce or eliminate the undesirable effects associated with the activity or proliferation of proliferating T cells. In inhibiting the activity or proliferation of a cell such inhibition may be direct or indirect.

The term “pharmaceutically acceptable salt” refers to an organic or inorganic moiety that carries a charge and that can be administered in association with a pharmaceutical agent, for example, as a counter-cation or counter-anion in a salt. Pharmaceutically acceptable cations are known to those of skilled in the art, and include but are not limited to sodium, potassium, calcium, zinc and quaternary amine. Pharmaceutically acceptable anions are known to those of skill in the art, and include but are not limited to chloride, acetate, citrate, bicarbonate and carbonate.

The term “pharmaceutically acceptable derivative” or “prodrug” refers to a derivative of the active compound that upon administration to the recipient, is capable of providing directly or indirectly, the parent compound or metabolite, or that exhibits activity itself. Prodrugs are included within the scope of the present invention.

The present application describes for the first time the immunomodulatory and immunopotentiating abilities of isoflavonoid compounds of formula I, exemplified by PXD. Such compounds are shown to have the ability to inhibit abnormal proliferation and/or activity of a subset of lymphocytes, typically T cells. The present application describes previously unexpected activities of the compounds disclosed herein and thus offers novel opportunities for the modulation of immune responses and novel opportunities for the treatment of, and augmentation of the treatment of, a variety of immune-related and immune-mediated disorders.

Accordingly, in one aspect the present invention provides for the use of a compound of formula I as described herein, or a pharmaceutically acceptable salt or prodrug thereof, as an immunomodulatory agent.

As exemplified herein, PXD is shown to inhibit plasma membrane electron transport, cell proliferation and cell survival, and induce apoptosis, in proliferating T cells whilst having no corresponding effect on resting T cells. PXD is also shown to prevent the ability of responder T cells to respond to external stimuli. In mixed lymphocyte reactions, proliferating allogeneic T cells were eliminated in the presence of PXD. Conversely, in such reactions non-proliferating T cells survived exposure to PXD and retained their ability to respond to external stimuli. Whilst not wishing to be limited by any particular mechanism of action it is proposed herein that PXD is a signal transduction regulator acting preferentially in abnormally dividing cells.

Accordingly, particular aspects and embodiments of the invention provide methods for inhibiting the activity and/or proliferation of T cells, and treating or preventing diseases and conditions associated with abnormal T cell proliferation or stimulation.

As used herein the term “associated with” as used with reference to diseases and conditions associated with abnormal T cell proliferation, means that a disease or condition may be caused by, may cause, or may otherwise be associated with abnormal T cell proliferation. Typically, in the context of the present invention, abnormal T cell proliferation means abnormally rapid proliferation. Such diseases and conditions include, by way of non-limiting example, T cell leukemias, autoimmune diseases, chronic viral infections such as HBV, and transplant or graft rejections such as graft versus host disease. Autoimmune diseases include, but are not limited to, cirrhosis, psoriasis, lupus, rheumatoid arthritis, colitis, diabetes mellitus, Addison's disease, infectious mononucleosis, Sézary's syndrome and Epstein-Barr virus infection. Those skilled in the art will appreciate that a wide range of diseases and conditions are associated with abnormal T cell proliferation and the present invention finds applicability in the treatment of any such disease or condition.

Also provided herein are methods for augmenting existing treatment regimes for subjects suffering from diseases or conditions associated with abnormal T cell proliferation or stimulation, the methods comprising administering to subjects in need thereof an immunomodulating effective amount of a compound of formula I as described herein or a pharmaceutically acceptable salt or prodrug thereof.

Thus, it is contemplated that compounds as described herein may be used in conjunction with existing therapeutic treatments for a range of diseases and conditions where a reduction in proliferating T cell activity and/or proliferation would be of benefit. That is, the administration of an immunomodulating effective amount of a compound described herein may improve the ability of a patient to respond to an existing treatment for the disease or condition suffered by the patient.

Compounds which find application in accordance with embodiments of the present invention are of the general formula (I):

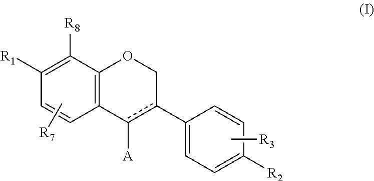

wherein
R₁ is hydroxy, alkoxy, halo or OC(O)R₉,
R₂ and R₃ are independently hydrogen, hydroxy, alkoxy, alkyl, halo or OC(O)R₉,
A is hydrogen or optionally substituted phenyl of the formula

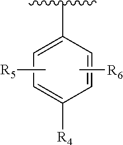

R₄, R₅ and R₆ are independently hydrogen, hydroxy, alkoxy, alkyl, amino, alkylamino, dialkylamino or OC(O)R₉,
R₇ and R₈ are independently hydrogen, hydroxy, alkyl, alkoxy or halo,
R₉ is hydrogen, alkyl, aryl, arylalkyl or amino, and
the drawing “ [symbol] ” represents a single bond or a double bond,
   or a pharmaceutically acceptable salt or prodrug thereof.

In an embodiment, alkyl is C₁₋₆-alkyl, C₁₋₄-alkyl, methyl, ethyl, propyl, isopropyl or tert-butyl. In a particular embodiment, alkyl is methyl.

In an embodiment, alkoxy is C₁₋₆-alkoxy, C₁₋₄-alkoxy, methoxy or ethoxy. In a particular embodiment alkoxy methoxy.

In an embodiment, halo is fluoro, chloro, bromo or iodo. In a particular embodiment, halo is chloro or bromo.

In an embodiment, aryl is phenyl, biphenyl or naphthyl optionally substituted by one or more C₁-C₄-alkyl, hydroxy, C₁-C₄-alkoxy, carbonyl, C₁-C₄-alkoxycarbonyl, C₁-C₄-alkylcarbonyloxy, nitro or halo. In a particular embodiment aryl is phenyl optionally substituted by methyl, hydroxy or methoxy.

In an embodiment, arylalkyl is benzyl optionally substituted by one or more C₁-C₄-alkyl, hydroxy, C₁-C₄-alkoxy, nitro or halo. In a particular embodiment arylalkyl substituted by methyl, hydroxy or methoxy.

In accordance with embodiments of the invention, in compounds of formula (I), the substitution pattern of R₂ and R₃ may be selected from:

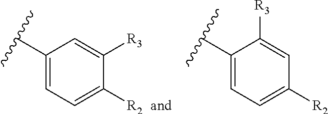

According to particular embodiments, in compounds of formula (I):
R₁ is hydroxy, C₁₋₄-alkoxy or OC(O)R₉,
one of R₂ and R₃ is hydrogen, hydroxy, C₁₋₄-alkoxy, halo or OC(O)R₉, and the other of R₂ and R₃ is hydroxy, C₁₋₄-alkoxy, halo or OC(O)R₉,
R₇ is hydrogen,
R₉ is hydrogen, hydroxy, C₁₋₄-alkyl or halo, and
R₉ is C₁₋₄-alkyl, phenyl or benzyl,
   or a pharmaceutically acceptable salt or prodrug thereof.

According to particular embodiments, in compounds of formula (I):
R₁ is hydroxy, methoxy or acetyloxy,
one of R₂ and R₃ is hydrogen, hydroxy, methoxy, bromo, chloro or acetyloxy, and the other of R₂ and R₃ is hydroxy, methoxy, bromo, chloro or acetyloxy, and
R₈ is hydrogen, hydroxy, methyl, methoxy, bromo or chloro,
   or a pharmaceutically acceptable salt or prodrug thereof.

Also according to particular embodiments in compounds of formula (I):
R₁ is hydroxy,
one of R₂ and R₃ is hydrogen, hydroxy or methoxy, and the other of R₂ and R₃ is hydroxy or methoxy, and
R₈ is hydrogen or methyl,
   or a pharmaceutically acceptable salt or prodrug thereof.

In a particular embodiment of the present invention the drawing “ [symbol] ” represents a double bond, and/or A is hydrogen. Thus according to a particular embodiment, compounds useful in the present invention may be of the general formula (I-a):

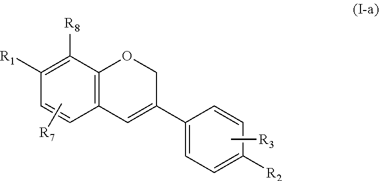

wherein R₁, R₂, R₃, R₇ and R₈ are as defined above.

Compounds of formula (I-a) may be selected from:
Isoflav-3-en-4′,7-diol (Cpd. 1);
4′-Methoxyisoflav-3-en-7,8-diol (Cpd. 2);
8-Methylisoflav-3-en-4′,7-diol (Cpd. 3);
Isoflav-3-en-7-ol (Cpd. 4);
Isoflav-3-en-3′,7-diol (Cpd. 5);
Isoflav-3-en-4′,7,8-triol (Cpd. 6);
8-Methylisoflav-3-en-3′,7-diol (Cpd. 7);
3′-Methoxy-8-methylisoflav-3-en-4′,7-diol (Cpd. 8);
3′-Methoxyisoflav-3-en-4′,7-diol (Cpd. 9);
3′-Methoxyisoflav-3-en-7-ol (Cpd. 10);
8-Methylisoflav-3-en-7-ol (Cpd. 11);
3′,4′-Dimethoxyisoflav-3-en-7-ol (Cpd. 12);
3′,4′-Dimethoxy-8-methylisoflav-3-en-7-ol (Cpd. 13);
8-Bromoisoflav-3-en-4′,7-diol (Cpd. 14);
Isoflav-3-en-4′,5,7-triol (Cpd. 15);
4′-Bromoisoflav-3-en-7-ol (Cpd. 16);

While not limited thereto, as exemplified herein, Cpd. (1) also known as dehydroequol or phenoxodiol, is a compound that finds a particular application in the present invention.

In another embodiment of the present invention the drawing “ [symbol] ” represents a single bond. A is optionally substituted phenyl. Thus according to a particular embodiment, compounds useful in the present invention may be of the general formula (I-b):

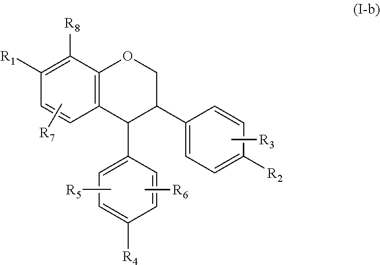

wherein R₁, R₂, R₃, R₄, R₅, R₆, R₇ and R₈ are as defined above.

In compounds of formula (I-b) the substitution pattern of R₄, R₅ and R₆ may be selected from:

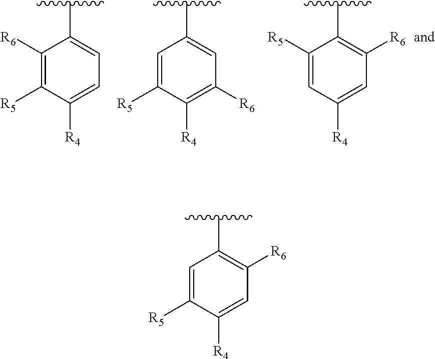

According to particular embodiments in compounds of formula (I-b):
R₄,R₅ and R₆ are independently hydrogen, hydroxy, C₁₋₄-alkoxy, C₁₋₄-alkyl, amino, OC(O)R₉, and
R₉ is C₁₋₄-alkyl, phenyl or benzyl,
   or a pharmaceutically acceptable salt or prodrug thereof.

According to particular embodiments in compounds of formula (I-b):
R₄ is hydrogen, hydroxy, methoxy, amino or acetyloxy, and
R₅ and R₆ are independently hydrogen, hydroxy, methoxy, amino or acetyloxy,
   wherein at least one of R₄, R₅ and R₆ are not hydrogen,
   or a pharmaceutically acceptable salt or prodrug thereof.

According to particular embodiments in compounds of formula (I-b):
one of R₄ and R₅ is hydrogen, hydroxy, methoxy or amino, and the other of R₄ and R₅ is hydroxy, methoxy or amino, and
R₆ is hydrogen,
   or a pharmaceutically acceptable salt or prodrug thereof.

According to particular embodiments in compounds of formula (I-b):
R₄ is methoxy, and
R₅ is hydrogen,
   or a pharmaceutically acceptable salt or prodrug thereof.

Compounds of formula (I-b) where R₈ is hydrogen include:
3-(4-Hydroxyphenyl)-4-(4-methoxyphenyl)chroman-7-ol (Cpd. 17);
3-(4-Hydroxyphenyl)-4-phenylchroman-7-ol (Cpd. 18);
3-(4-Hydroxyphenyl)-4-(3-methoxyphenyl)chroman-7-ol (Cpd. 19);
3-(3,4-Dimethoxyphenyl)-4-(4-methoxyphenyl)chroman-7-ol (Cpd. 20);
3-(4-Hydroxyphenyl)-4-(4-methylphenyl)chroman-7-ol (Cpd. 21);
3-(4-Methoxyphenyl)-4-(4-methoxyphenyl)-7-methoxychroman (Cpd. 22);
3-(4-Hydroxyphenyl)-4-(2,6-dimethoxy-4-hydroxyphenyl)chroman-7-ol (Cpd. 23);
3-(4-Hydroxyphenyl)-4-(2-hydroxyphenyl)chroman-7-ol (Cpd. 24);
3-(4-Hydroxyphenyl)-4-(3-acyl-2-hydroxy-4-methoxyphenyl)chroman-7-ol (Cpd. 25);
3-(3-Hydroxyphenyl)-4-(3-methoxyphenyl)chroman-7-ol (Cpd. 26);
3-(4-Hydroxyphenyl)-4-(4-hydroxyphenyl)chroman-7-ol (Cpd. 27);
3-(4-Bromophenyl)-4-(4-methoxyphenyl)chroman-7-ol (Cpd. 28);
3-(4-Hydroxyphenyl)-4-(3-methoxyphenyl)chroman-7-ol (Cpd. 29);
3-(4-Hydroxyphenyl)-4-(3-aminophenyl)chroman-7-ol (Cpd. 30); and
3-(4-Hydroxyphenyl)-4-(4-phenoxyphenyl)chroman-7-ol (Cpd 31);
   or pharmaceutically acceptable salts thereof.

Compounds of formula (I-b) where R₈ is methyl include:
3-(4-Hydroxyphenyl)-4-(4-methoxyphenyl)-8-methylchroman-7-ol (Cpd. 32);
3-(4-Methoxyphenyl)-4-(4-methoxyphenyl)-7-methoxy-8-methylchroman (Cpd. 33);
3-(3,4-Dimethoxyphenyl)-4-(4-methoxyphenyl)-8-methylchroman-7-ol (Cpd. 34);
3-(4-Methoxyphenyl)-4-(4-methoxyphenyl)-8-methylchroman-7-ol (Cpd. 35);
3-(4-Hydroxyphenyl)-4-(4-methoxyphenyl)-7-methoxy-8-methylchroman (Cpd. 36);
3-(3-Methoxyphenyl)-4-(4-methoxyphenyl)-8-methylchroman-7-ol (Cpd. 37);
3-(3,4-Dihydroxyphenyl)-4-(4-methoxyphenyl)-7-methoxy-8-methylchroman (Cpd. 38);
3-(3-Hydroxyphenyl)-4-(4-methoxyphenyl)-8-methylchroman-7-ol (Cpd. 39); and
3-(3,4-Dihydroxyphenyl)-4-(4-methoxyphenyl)-8-methylchroman-7-ol (Cpd. 40);
   or pharmaceutically acceptable salts thereof.

Compounds of formula (I-b) for use according to the invention have two chiral centres. All enantiomers and diastereoisomers including isolated or pairs of enantiomers or diastereoisomers as well as mixtures thereof in any proportions are contemplated for use in accordance with the invention. It will be clear to persons skilled in the art that the in compounds of formula (I-b) the aryl substituents on the heterocyclic ring can be cis- or trans-relative to each other.

In a particular embodiment the cis-isomer of Cpd. 17 or a pharmaceutically acceptable salt thereof is contemplated:

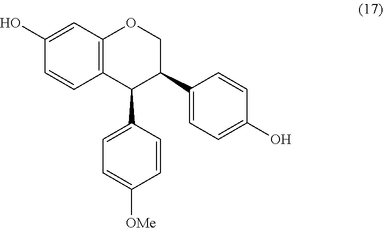

In a particular embodiment the cis-isomer of Cpd. 32 or a pharmaceutically acceptable salt thereof is contemplated:

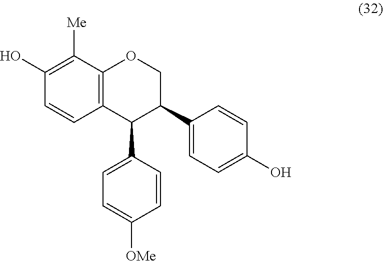

Similarly, Cpds. 18 to 31 and 33 to 40 in the cis-conformation are also contemplated in particular embodiments.

The term “alkyl” is taken to include straight chain and branched chain saturated alkyl groups of 1 to 6 carbon atoms, such as methyl, ethyl, propyl, isopropyl, butyl, isobutyl, secbutyl, tertiary butyl, pentyl and the like. The alkyl group more preferably contains from 1 to 4 carbon atoms, especially methyl, ethyl, propyl or isopropyl. The alkyl group or cycloalkyl group may optionally be substituted by one or more of fluorine, chlorine, bromine, iodine, carboxyl, C₁-C₄-alkoxycarbonyl, C₁-C₄-alkylaminocarbonyl, di-(C₁-C₄-alkyl)-amino-carbonyl, hydroxyl, C₁-C₄-alkoxy, formyloxy, C₁-C₄-alkylcarbonyloxy, C₁-C₄-alkylthio, C₃-C₆-cycloalkyl or phenyl. Typically the alkyl group does not bear any substituents.

The term “aryl” is taken to include phenyl, benzyl, biphenyl and naphthyl and may be optionally substituted by one or more C₁-C₄-alkyl, hydroxy, C₁-C₄-alkoxy, carbonyl, C₁-C₄-alkoxycarbonyl, C₁-C₄-alkylcarbonyloxy, nitro or halo.

The term “halo” is taken to include fluoro, chloro, bromo and iodo, preferably fluoro and chloro, more preferably fluoro. Reference to for example “haloalkyl” will include monohalogenated, dihalogenated and up to perhalogenated alkyl groups. Typical haloalkyl groups are, for example, trifluoromethyl and pentafluoroethyl.

In accordance with the present invention pharmaceutically acceptable salts and derivatives of the compounds disclosed herein may be employed. Pharmaceutically acceptable salts are well known to those skilled in the art and include those formed from: acetic, ascorbic, aspartic, benzoic, benzenesulphonic, citric, cinnamic, ethanesulphonic, fumaric, glutamic, glutaric, gluconic, hydrochloric, hydrobromic, lactic, maleic, malic, methanesulphonic, naphthoic, hydroxynaphthoic, naphthalenesulphonic, naphthalenedisulphonic, naphthaleneacrylic, oleic, oxalic, oxaloacetic, phosphoric, pyruvic, p-toluenesulphonic, tartaric, trifluoroacetic, triphenylacetic, tricarballylic, salicylic, sulphuric, sulphamic, sulphanilic and succinic acid.

Pharmaceutically acceptable derivatives are well known to those skilled in the are and include solvates, pharmaceutically active esters, prodrugs or the like. This also includes derivatives with physiologically cleavable leaving groups that can be cleaved in vivo to provide the compounds of the invention or their active moiety. The leaving groups may include acyl, phosphate, sulfate, sulfonate, and preferably are mono-, di- and per-acyl oxy-substituted compounds, where one or more of the pendant hydroxy groups are protected by an acyl group, preferably an acetyl group. Typically acyloxy substituted compounds of the invention are readily cleavable to the corresponding hydroxy substituted compounds.

Compounds of formula I as described herein are believed to have favourable active profiles and good bioavailability. These compounds are described in International Patent Applications PCT/AU2005/001435 (published as WO 2006/032085), PCT/AU2005/001436 (published as WO 2006/032086) and PCT/A000/00103 (published as WO 00/49009), the disclosures of which are incorporated herein by reference.

According to the methods of present invention isoflavonoid compounds disclosed herein and compositions comprising such compounds may be administered by any suitable route, systemically, regionally or locally. The particular route of administration to be used in any given circumstance will depend on a number of factors, including the nature of the condition to be treated, the severity and extent of the condition, the required dosage of the particular compound to be delivered and the potential side-effects of the compound. For example, in circumstances where it is required that appropriate concentrations of the desired compound are delivered directly to the site in the body to be treated, administration may be regional rather than systemic. Regional administration provides the capability of delivering very high local concentrations of the desired compound to the required site and thus is suitable for achieving the desired therapeutic or preventative effect whilst avoiding exposure of other organs of the body to the compound and thereby potentially reducing side effects.

By way of example, administration according to embodiments of the invention may be achieved by any standard routes, including intracavitary, intravesical, intramuscular, intraarterial, intravenous, intraocular, subcutaneous, topical or oral.

In employing methods of the invention, isoflavonoid compounds may be formulated in pharmaceutical compositions. Suitable compositions may be prepared according to methods which are known to those of ordinary skill in the art and may include a pharmaceutically acceptable diluent, adjuvant and/or excipient. The diluents, adjuvants and excipients must be “acceptable” in terms of being compatible with the other ingredients of the composition, and not deleterious to the recipient thereof. The diluent, adjuvant or excipient may be a solid or a liquid, or both, and may be formulated with the compound as a unit-dose, for example, a tablet, which may contain from 0.5% to 59% by weight of the active compound, or up to 100% by weight of the active compound. One or more active compounds may be incorporated in the formulations of the invention, which may be prepared by any of the well known techniques of pharmacy consisting essentially of admixing the components, optionally including one or more accessory ingredients.

Examples of pharmaceutically acceptable diluents are demineralised or distilled water; saline solution; vegetable based oils such as peanut oil, safflower oil, olive oil, cottonseed oil, maize oil, sesame oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrridone; agar; carrageenan; gum tragacanth or gum acacia, and petroleum jelly. Typically, the carrier or carriers will form from 1% to 99.9% by weight of the compositions.

Formulations suitable for oral administration may be presented in discrete units, such as capsules, sachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above). In general, the formulations of the invention are prepared by uniformly and intimately admixing the active compound with a liquid or finely divided solid carrier, or both, and then, if necessary, shaping the resulting mixture such as to form a unit dosage. For example, a tablet may be prepared by compressing or moulding a powder or granules containing the active compound, optionally with one or more accessory ingredients. Compressed tablets may be prepared by compressing, in a suitable machine, the compound of the free-flowing, such as a powder or granules optionally mixed with a binder, lubricant, inert diluent, and/or surface active/dispersing agent(s). Moulded tablets may be made by moulding, in a suitable machine, the powdered compound moistened with an inert liquid binder.

Solid forms for oral administration may contain binders acceptable in human and veterinary pharmaceutical practice, sweeteners, disintegrating agents, diluents, flavourings, coating agents, preservatives, lubricants and/or time delay agents. Suitable binders include gum acacia, gelatine, corn starch, gum tragacanth, sodium alginate, carboxymethylcellulose or polyethylene glycol.

Suitable sweeteners include sucrose, lactose, glucose, aspartame or saccharine. Suitable disintegrating agents include corn starch, methylcellulose, polyvinylpyrrolidone, guar gum, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

Liquid forms for oral administration may contain, in addition to the above agents, a liquid carrier. Suitable liquid carriers include water, oils such as olive oil, peanut oil, sesame oil, sunflower oil, safflower oil, arachis oil, coconut oil, liquid paraffin, ethylene glycol, propylene glycol, polyethylene glycol, ethanol, propanol, isopropanol, glycerol, fatty alcohols, triglycerides or mixtures thereof.

Formulations suitable for buccal (sublingual) administration include lozenges comprising the active compound in a flavoured base, usually sucrose and acacia or tragacanth; and pastilles comprising the compound in an inert base such as gelatin and glycerin or sucrose and acacia.

Compositions of the present invention suitable for parenteral administration typically conveniently comprise sterile aqueous preparations of the active compounds, which preparations may be isotonic with the blood of the intended recipient. These preparations are typically administered intravenously, although administration may also be effected by means of subcutaneous, intramuscular, or intradermal injection. Such preparations may conveniently be prepared by admixing the compound with water or a glycine buffer and rendering the resulting solution sterile and isotonic with the blood. Injectable formulations according to the invention generally contain from 0.1% to 60% w/v of active compound(s) and are administered at a rate of 0.1 ml/minute/kg or as appropriate.

Formulations for infusion, for example, may be prepared employing saline as the carrier and a solubilising agent such as a cyclodextrin or derivative thereof. Suitable cyclodextrins include α-cyclodextrin, β-cyclodextrin, γ-cyclodextrin, dimethyl-β-cyclodextrin, 2-hydroxyethyl-β-cyclodextrin, 2-hydroxypropyl-cyclodextrin, 3-hydroxypropyl-β-cyclodextrin and tri-methyl-β-cyclodextrin. More preferably the cyclodextrin is hydroxypropyl-β-cyclodextrin. Suitable derivatives of cyclodextrins include Captisol® a sulfobutyl ether derivative of cyclodextrin and analogues thereof as described in U.S. Pat. No. 5,134,127.

Formulations suitable for rectal administration are typically presented as unit dose suppositories. These may be prepared by admixing the active compound with one or more conventional solid carriers, for example, cocoa butter, and then shaping the resulting mixture.

Formulations or compositions suitable for topical administration to the skin may take the form of an ointment, cream, lotion, paste, gel, spray, aerosol, or oil. Carriers which may be used include Vaseline, lanoline, polyethylene glycols, alcohols, and combination of two or more thereof. The active compound is generally present at a concentration of from 0.1% to 0.5% w/w, for example, from 0.5% to 2% w/w. Examples of such compositions include cosmetic skin creams.

Formulations suitable for inhalation may be delivered as a spray composition in the form of a solution, suspension or emulsion. The inhalation spray composition may further comprise a pharmaceutically acceptable propellant such as carbon dioxide or nitrous oxide or a hydrogen containing fluorocarbon such as 1,1,1,2-tetrafluoroethane, 1,1,1,2,3,3,3-heptafluoro-n-propane or mixtures thereof.

Formulations suitable for transdermal administration may be presented as discrete patches adapted to remain in intimate contact with the epidermis of the recipient for a prolonged period of time. Such patches suitably contain the active compound as an optionally buffered aqueous solution of, for example, 0.1 M to 0.2 M concentration with respect to the said active compound. Formulations suitable for transdermal administration may also be delivered by iontophoresis (see, for example, Pharmaceutical Research 3 (6), 318 (1986)) and typically take the form of an optionally buffered aqueous solution of the active compound. For example, suitable formulations may comprise citrate or bis/tris buffer (pH 6) or ethanol/water and contain from 0.1 M to 0.2 M active ingredient.

The active compounds may be provided in the form of food stuffs, such as being added to, admixed into, coated, combined or otherwise added to a food stuff. The term food stuff is used in its widest possible sense and includes liquid formulations such as drinks including dairy products and other foods, such as health bars, desserts, etc. Food formulations containing compounds of the invention can be readily prepared according to standard practices.

According to the present invention, compounds and compositions may be administered either therapeutically or preventively. In a therapeutic application, compounds and compositions are administered to a patient already suffering from a disease or disorder or experiencing symptoms, in an amount sufficient to cure or at least partially arrest the disease or disorder, symptoms and/or any associated complications. The compound or composition should provide a quantity of the active compound sufficient to effectively treat the patient.

The effective dose of the administered compound for any particular subject will depend upon a variety of factors including: the type of condition being treated and the stage of the condition; the activity of the compound employed; the composition employed; the age, body weight, general health, sex and diet of the patient; the time of administration; the route of administration; the rate of sequestration of compounds; the duration of the treatment; drugs used in combination or coincidental with the treatment, together with other related factors well known in medicine.

One skilled in the art would be able, by routine experimentation, to determine an effective, non-toxic dosage which would be required to treat applicable conditions. These will most often be determined on a case-by-case basis. By way of example only, an effective dosage may be expected to be in the range of about 0.0001 mg to about 1000 mg per kg body weight per 24 hours; typically, about 0.001 mg to about 750 mg per kg body weight per 24 hours; about 0.01 mg to about 500 mg per kg body weight per 24 hours; about 0.1 mg to about 500 mg per kg body weight per 24 hours; about 0.1 mg to about 250 mg per kg body weight per 24 hours; about 1.0 mg to about 250 mg per kg body weight per 24 hours; or about 10 mg to about 200 mg per kg body weight per 24 hours.

Further, it will be apparent to those of ordinary skill in the art that the optimal quantity and spacing of individual dosages will principally be determined by the nature and extent of the condition being treated, the form, route and site of administration, and the individual being treated. Suitable conditions can be determined by conventional techniques.

It will also be apparent to those of ordinary skill in the art that the optimal course of treatment, such as, the number of doses of the composition given per day for a defined number of days, can be ascertained by those skilled in the art using conventional course of treatment determination tests.

In accordance with the methods of the invention, isoflavonoid compounds or pharmaceutically acceptable derivatives, prodrugs or salts thereof can be co-administered with other active agents that do not impair the desired action, or with agents that supplement the desired action. The particular agent(s) used will depend on a number of factors and will typically be tailored to the disease or disorder to be treated. The co-administration of agents may be simultaneous or sequential. Simultaneous administration may be effected by the compounds being formulated in a single composition, or in separate compositions administered at the same or similar time. Sequential administration may be in any order as required.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The present invention will now be described with reference to the following specific examples, which should not be construed as in any way limiting the scope of the invention.

### EXAMPLES

General Methods

Materials

Concentrated blood bags (50 mL) from healthy volunteers were provided by the Red Cross Blood Bank (Melbourne, Australia). Bone marrow samples were obtained from the tissue bank at the Peter MacCallum Cancer Institute after obtaining ethics approval from the Peter MacCallum Tissue Research Management Committee (project number 07/14).

2-(4-iodophenyl)-3-(4-nitrophenyl)-5-(2,4-disulfophenyl)-2H-tetrazolium monosodium salt (WST-1) and 1-methoxyphenazine methylsulfate (1 mPMS) were purchased from Dojindo Laboratories (Kumamoto, Japan). PXD was obtained from Novogen Inc (NSW, Australia). Human anti-CD3 mAb, human anti-CD28 mAb, propidium iodide (PI), APC-labelled mouse anti-human anti-CD3 mAb and FITC-labelled annexin V (AV) were from Pharmingen (Becton Dickinson, North Ryde, Australia). Human recombinant IL-2 was obtained from the Biological Resources Branch Preclinical Repository, NCI (Frederick, Md.). Unless otherwise stated all other reagents were from Sigma (St. Louis, Mo., U.S.A.). PXD was stored in solid form under nitrogen gas to prevent oxidation and dissolved prior to each experiment in DMSO at 10000× or 1000× the final concentration before being diluted in Hanks Balanced Salts Solution (HBSS) or RPMI-1640 medium (GIBCO-BRL, Grand Island, N.Y.), supplemented with 10% (v/v) fetal calf serum (FCS). Controls consisted of DMSO at the same concentrations as those used in the PXD treatment.

Cell lines were grown in RPMI-1640 medium (GIBCO-BRL, Grand Island, N.Y.) supplemented with 5% (v/v) fetal calf serum, 2 mM glutamate, 25 μg/mL penicillin, 25 μg/mL streptomycin, 50 μg/mL uridine and 1 mM pyruvate to densities of 1-2×10⁶ cells/mL (exponential stage), at 37° C. in a humidified incubator maintained at 5% CO₂.

Collection, Isolation and Storage of PBMC from Buffy Coats

PBMC were isolated from buffy coats of blood bags from healthy volunteers using Ficoll gradients. PBMC were resuspended in RPMI-1640 medium (GIBCO-BRL, Grand Island, N.Y.) supplemented with 10% (v/v) FCS and viable cells counted by Trypan blue exclusion. PBMC were stored at 20-30×10⁶ cells/vial in 1.5 mL 90% FCS+10% DMSO in liquid nitrogen. The frequency of different cell types in the PBMC samples used for these experiments was determined by FACS analysis as follows: 70-80% CD3⁺ (T lymphocytes), 5-15% CD19⁺ (B lymphocytes), 10-20% CD14⁺ (monocytes), 5-10% CD56⁺ (NK cells), <5% CD15⁺ (neutrophils). The CD3⁺ cell population consisted of 30-40% CD8⁺ and 60-70% CD4⁺ T cells.

In Vitro Activation of PBMC (T Cells)

Immediately prior to experiments, frozen PBMC were thawed, centrifuged at 1400 rpm in a Multifuge 3 s (Heraeus) for 4 min, washed in RPMI+10% FCS and resuspended in T cell medium (RPMI+10% FCS+10 μM 2-mercaptoethanol (Sigma), 1×glutamine, 1×non-essential amino acids (Gibco)) at 2×10⁶ cells/mL. Cells (2×10⁵) were activated by adding 10 μg/mL anti-CD3 and 5 μg/mL anti-CD28 and 20 U/mL IL-2 per well. Cells were incubated at 37° C. in a humidified incubator maintained at 5% CO₂ for 4 to 5 days until large numbers of large spherical aggregates or “bursts” of lymphocytes were observed. Activated proliferating T cells were pooled from the wells for further experiments. Resting T cells were incubated for the same length of time but in the presence of 20 U/mL IL-2 only.

Mixed Lymphocyte Reactions (MLR)

Mixed lymphocyte reactions were established using unrelated, HLA-mismatched healthy donor blood samples as donor/recipient pairs. Stimulator PBMC were γ-irradiated with 30 Gy as described previously (Zenhausen et al., 2007). Responder PBMC were washed twice in PBS, 5,6-carboxy-succinimidyl-fluorescein-ester (CSFE)-labelled (1.25 μM, Sigma) at room temperature for 5 minutes, and washed twice in PBS/10% FCS. Two hundred thousand stimulator and responder cells were added per well at a 1:1 ratio in a 96 U-well plate in T cell medium. Stimulator and responder cells were also plated alone, and all cells were cultured in a 5% CO2, 37° C. humidified incubator. Viability, CFSE fluorescence and cell surface antigen expression were determined by flow cytometry. Cells were washed twice in FACS buffer (PBS, 2% FCS), and resuspended in 50 μL/5×10⁵ cells and analysed on a Becton Dickinson LSRII FACS analyser using FlowJo (TreeStar) software.

Isolation of Leukemic Blasts from Bone Marrow Samples of AML and ALL Patients

Bone marrow samples from AML (n=22) and ALL (n=8) patients, containing more than 80% blasts, were selected from the tissue bank. Bone marrow samples were thawed, centrifuged at 1400 rpm in a Multifuge 3 s (Heraeus) for 4 min, washed twice in RPMI and resuspended in RPMI+10% FCS. Aliquots were incubated for 24 h in the presence of 0.1% DMSO (control) or 10 μM PXD and examined for morphology and viability. Percentage apoptosis was determined by AV/PI staining as [% viable blasts after PXD treatment]/[% viable blasts in control sample].

PMET Activity Measured by WST-1/PMS Reduction

WST-1/PMS reduction rates were measured in a microplate format as described previously (Berridge and Tan, 1998). Briefly, exponentially growing cells were centrifuged at 1400 rpm in a Multifuge 3 s (Heraeus) for 4 min, washed and resuspended in HBSS buffer. For each assay, 50 μL of a 2×10⁶ cells/mL cell suspension was pipetted into flat-bottomed microplate wells containing 50 μL of the inhibitor/buffer solution, resulting in a final concentration of 1×10⁶ cells/mL. Dye reduction was initiated by adding 10 μL of a 10× stock solution of WST-1/PMS in milliQ water (final concentrations of 500 μM WST-1 and 20 μM PMS). WST-1 reduction was measured in real time at 450 nm over 30-60 min in a BMG FLUOstar OPTIMA plate reader.

Cell Proliferation Measured by MTT Reduction

MTT reduction was measured as previously described (Berridge et al., 1996) in a microplate format as follows: exponentially growing cells were centrifuged at 1400 rpm in a Multifuge 3 s (Heraeus) for 4 min, washed and resuspended in HBSS buffer. For each assay, 50 μL of a 2×10⁶ cells/mL cell suspension was pipetted into flat-bottomed microplate wells containing 50 μL of the inhibitor/buffer solution, resulting in a final concentration of 1×10⁶ cells/mL. Following 48 h incubation, dye reduction was initiated by adding 10 μL of 5 mg/mL MTT to each well. After 2 h, 100 μL of lysing buffer was added and formazan crystals dissolved by manual pipetting using a multichannel pipette before measuring A570 in a BMG FLUOstar OPTIMA plate reader.

Cell Viability Measured by Trypan Blue Exclusion

Proliferating and resting PBMCs were centrifuged at 1400 rpm in a Multifuge 3 s (Heraeus) for 4 min at room temperature, resuspended in fresh T cell medium in 96 U-well plates (200 μL per well at densities of 2×10⁶ cells/mL) in the presence of PXD or 0.1% DMSO and incubated in a 5% CO₂, 37° C. humidified incubator. Viable cells, as determined by Trypan blue exclusion, were counted in a Neubauer haemocytometer every 24 h for several days.

Annexin V/Propidium Iodide Staining

Cells were centrifuged at 1400 rpm in a Multifuge 3 s (Heraeus) for 4 min at room temperature, washed in phosphate buffered saline solution, pH 7.3 (PBS) and resuspended in Annexin V binding buffer. Small aliquots (0.5-1×10⁶ cells) were transferred to 1.5 mL tubes and spun at 1400 rpm in a Multifuge 3 s (Heraeus) for 4 min. Most supernatant was removed and 5 μL PI and 5 μL FITC-labelled AV was added to the wet pellets which were vortexed. After 30 min in the dark on ice, 500 μl of AV binding buffer was added, the cells were centrifuged for 2 min at 1400 rpm, washed once with AV binding buffer and resuspended in 300 μL of AV binding buffer in FACS tubes. Staining was analyzed by flow cytometry using a Becton Dickinson Canto II FACS analyser using FlowJo (TreeStar) software.

### Example 1

### Effect of PXD on Viability of Rapidly Proliferating T Cells

PXD Inhibits PMET, Cell Proliferation and Viability of Rapidly Proliferating T Cells

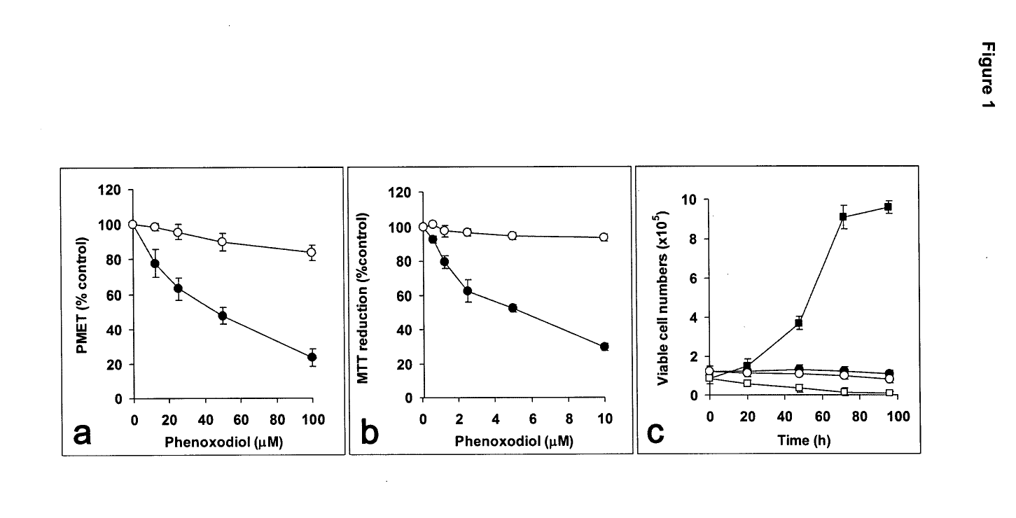

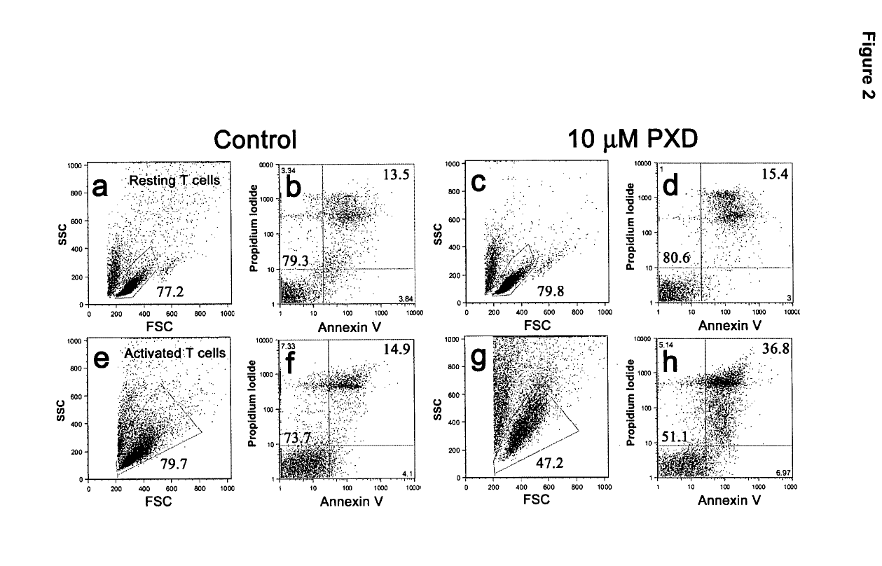

The effect of PXD on PMET (FIG. 1A) was investigated by measuring reduction of the cell impermeable tetrazolium dye, WST-1, in the presence of its obligate intermediate electron acceptor, 1 mPMS (WST-1/PMS reduction). Exposure to PXD inhibited PMET of proliferating T cells (IC₅₀ of 46 μM) but had only a minor inhibitory effect on resting T cells (>200 μM). The effect of PXD on proliferation (FIG. 1B) was determined by measuring intracellular reduction of the tetrazolium salt, MTT. MTT reduction was inhibited in the presence of PXD in proliferating T cells (IC₅₀=5.4 μM), but not in resting T cells (IC₅₀>200 μM). Under the experimental conditions used, proliferating T cells had a cycling time of 21 h, whereas resting T cells did not proliferate but remain viable, regardless of the presence of 10 μM PXD (FIG. 1C). However, the viability of proliferating T cells was severely compromised by incubation with 10 μM PXD.

PXD Causes Apoptosis of Proliferating T Cells

The extent of apoptosis in resting and proliferating T cells was determined after 24 h exposure to 10 μM PXD (FIG. 2) by AV/PI staining. The scatter plots show that untreated large proliferating T cells blasts (FIG. 2E) underwent apoptosis after treatment with PXD (FIG. 2H). Resting T cells were not affected (FIG. 2D). The percentage of viable proliferating T cells dropped from 74% (FIG. 2F) to 51% after exposure to 10 μM PXD for 24 h (FIG. 2H), whilst the percentage of viable resting T cells was unaltered (FIGS. 2B and D).

Brief Exposure to PXD is Enough to Kill Proliferating T Cells

In order to investigate whether cells need to be exposed continually to PXD for its effects to manifest, proliferating T cells were exposed to 10 μM PXD for different periods of time, washed cells twice in RPMI and incubated for a further 24 h in fresh T cell medium. Brief exposure to 10 μM PXD of one minute induced apoptosis in proliferating T cells to the same extent as seen after 24 h exposure to PXD. Pre-incubation of PXD in 90% FCS or 90% human serum did not affect the ability of PXD to kill proliferating T cells (Table 1).

TABLE 1
Effect exposure time to PXD on survival of proliferating T cells

| | % Viable cells after different | | |
|---|---|---|---|
| | exposure times** | | |
| Pre-incubation* conditions* | 1 min | 10 min | 24 h |
| 10% FCS | 64 ± 4.6 | 60 ± 7.1 | 70 ± 7.1 |
| 90% FCS | 66 ± 4.2 | 62 ± 5.7 | 69.0 ± 7.2 |
| 90% Human Serum | 69.0 ± 4.4 | 72 ± 7.3 | 73.5 ± 9.2 |

*100 μM PXD was pre-incubated in different concentrations of serum for 1 h at 37° C., washed twice in RPMI and added to proliferating T cells to a final concentration of 10 μM. Cells were then incubated for a further 24 h.
**Calculated as [% (AV-PI-) after exposure to PXD]/[% (AV-PI-) of cells exposed to 0.1% DMSO].
Results are presented as the average ± SEM of at least two separate experiments.

PXD Eliminates Proliferating Responder T Cells in HLA-Mismatched MLR

The ability of PXD to induce apoptosis in proliferating T cells was further tested in a HLA-mismatched MLR. CFSE labelled responder cells were mixed with HLA-mismatched γ-irradiated stimulator cells in the presence of 0.1% DMSO (control) and 10 μM PXD on day 0. Responder cells were analysed for proliferation (CFSEIO) and viability (AV⁻) at day 8 to allow exposure of proliferating responder cells to the drug for several days. Strong activation and proliferation of allogeneic T cells (CD3⁺CFS^{lo}AV⁻ population) was seen in the control MLR (46.5%, FIG. 3D) but not in the PXD treated MLR (2%, FIG. 3B). In contrast, a resting responder cell population (CD3⁺CFSE^{hi}AV⁻) was present in both control and PXD treated MLR.

### Example 2

### Effect of PXD on Unstimulated and Responder T Cells

Unstimulated T Cells Respond to Foreign Antigen After Transient Exposure to PXD

The inventors next determined whether a transient exposure to PXD affected the ability of unstimulated T cells to respond normally to foreign antigen. Unstimulated T cells were incubated with 10 μM PXD for 24 h, washed twice and stimulated by adding HLA-mismatched γ-irradiated stimulator cells. FACS analysis 8 days later showed that transient exposure to PXD by unstimulated responder T cells did not affect their subsequent activation and proliferation, as evidenced by similar sized CD3⁺CFSE^{lo}AV⁻ populations (62.6% in the control MLR and 56.2% in the PXD treated MLR) (FIG. 4).

Resting Responder T Cells can be Restimulated in a Third Party MLR

The experiment described above was expanded by determining the effect of PXD exposure on responder T cells that had previously been exposed to, but not activated in a MLR. Two consecutive sets of MLRs were set up, mixing responder T cells and γ-irradiated stimulator cells on day 0, adding 10 μM PXD or 0.1% DMSO on day 5, and analysing proliferation and viability of responder T cells on day 8. The viable resting (CD3⁺CFSE^{hi}AV⁻) populations were then sorted by FACS and stimulated in a subsequent set of third party MLRs in the absence of PXD. During the second MLR, stimulated T cells proliferated (CD3⁺CSFE^{lo} population in FIG. 5D) but not unstimulated T cells (FIG. 5B), suggesting that PXD affects neither the viability nor the functionality of non-proliferating T cells.

### Example 3

### PXD Causes Apoptosis in Leukemic Cell Lines and Leukemic Blasts from Bone Marrow Samples

Although PXD has been shown to cause apoptosis in a number of cell lines, clinical trials have so far focussed on solid cancers, including ovarian, breast and prostate cancer and melanoma. The inventors have previously shown that PXD killed AML-derived HL60 cells and here this finding was extended to a panel of haematological cancers as well as to a number of primary leukemic blasts from bone marrow samples of ALL and AML patients (FIG. 6). The specific characteristics of these clinical samples are described in Table 2. The sensitivity to PXD varied between cell lines and primary cells. Interestingly, ALL blasts were more consistent in their response to PXD and significantly more sensitive than AML blasts (p=0.0002), which showed extensive variability in their sensitivity to PXD (FIG. 6).

TABLE 2
Characteristics of ALL and AML clinical samples

| Patient | Disease Specifics | % Viable Blasts | Timing |
|---|---|---|---|
| 1 | B cell ALL | 7 | R |
| 2 | B cell ALL | 14 | D |
| 3 | ALL unspec | 16 | R |
| 4 | T cell ALL | 19 | R |
| 5 | APML | 20 | D |
| 6 | biphenotypic AML | 23 | R |
| 7 | B cell ALL | 25 | R |
| 8 | B cell ALL | 29 | D |
| 9 | AML | 32 | R |
| 10 | AML M1 | 36 | R |
| 11 | B cell ALL | 37 | D |
| 12 | AML | 39 | D |
| 13 | B cell ALL | 40 | D |
| 14 | AML | 44 | D |
| 15 | AML M5 | 49 | D |
| 16 | AML | 55 | D |
| 17 | AML | 57 | R |
| 18 | APML | 58 | D |
| 19 | AML | 62 | R |
| 20 | AML M5 | 62 | R |
| 21 | AML M5 | 67 | D |
| 22 | AML | 71 | R |
| 23 | AML | 75 | R |
| 24 | AML | 88 | R |
| 25 | AML | 92 | R |
| 26 | AML | 95 | R |
| 27 | AML | 96 | D |
| 28 | AML | 97 | D |
| 29 | AML | 99 | R |
| 30 | AML | 99 | D |

R = sample taken at relapse.
D = sample taken at diagnosis

### REFERENCES

Alvero A B, Brown D, Montagna M, Matthews M, Mor G. (2007) Phenoxodiol-Topotecan Co-Administration Exhibit Significant Anti-Tumor Activity Without Major Adverse Side Effects. Cancer Biol Ther. 6:612-7.
Alvero A B, Kelly M, Rossi P, Leiser A, Brown D, Rutherford T, Mor G. (2008) Anti-tumor activity of phenoxodiol: from bench to clinic. Future Oncol. 4(4):475-82.
Berridge M V, Tan A S, McCoy K D, Wang R. (1996) The biochemical and cellular basis of cell proliferation asays that use tetrazolium salts. Biochemica. 4:15-20.
Berridge M V, Tan A S. (1998) Trans-plasma membrane electron transport: a cellular assay for NADH- and NADPH-oxidase based on extracellular, superoxide-mediated reduction of the sulfonated tetrazolium salt WST-1. Protoplasma. 205:74-82.
Choueiri T K, Mekhail T, Hutson T E, Ganapathi R, Kelly G E, Bukowski R M. (2006) Phase I trial of phenoxodiol delivered by continuous intravenous infusion in patients with solid cancer. Ann Oncol. 17:860-5.
de Souza P L, Liauw W, Links M, Pirabhahar S, Kelly G, Howes L G. (2006) Phase I and pharmacokinetic study of weekly NV06 (Phenoxodiol), a novel isoflav-3-ene, in patients with advanced cancer. Cancer Chemother Pharmacol. 58:427-33.
Zenhausen G, Gasser O, Saleh L, Villard J, Tiercy J-M, Hess C. (2007) Investigation of alloreactive NK cells in mixed lymphocyte reactions using paraformaldehyde-silenced target cells. J Immunol Methods. 321:196-199.

1. A method for inhibiting the proliferation and/or activity of proliferating T cells, the method comprising exposing the proliferating T cells to an effective amount of a compound of formula I 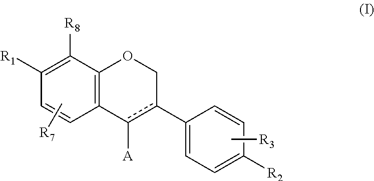
wherein
R₁ is hydroxy, alkoxy, halo or OC(O)R₉,
R₂ and R₃ are independently hydrogen, hydroxy, alkoxy, alkyl, halo or OC(O)R₉,
A is hydrogen or optionally substituted phenyl of the formula 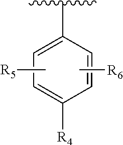
R₄, R₅ and R₆ are independently hydrogen, hydroxy, alkoxy, alkyl, amino, alkylamino, dialkylamino or OC(O)R₉,
R₇ and R₈ are independently hydrogen, hydroxy, alkyl, alkoxy or halo,
R₉ is hydrogen, alkyl, aryl, arylalkyl or amino, and
the drawing “ [symbol] ” represents a single bond or a double bond,
or a pharmaceutically acceptable salt or prodrug thereof.

2. The method of claim 1, wherein the compound is selected from isoflav-3-en-4′,7-diol, 3-(4-hydroxyphenyl)-4-(4-methoxyphenyl)chroman-7-ol, 3-(4-hydroxyphenyl)-4-(4-hydroxyphenyl)chroman-7-ol and 3-(4-hydroxyphenyl)-4-(4-methoxyphenyl)-8-methylchroman-7-ol.

3. The method of claim 2 wherein the compound is 3-(4-hydroxyphenyl)-4-(4-hydroxyphenyl)chroman-7-ol.

4. (canceled)

5. The method of claim 1, wherein the compound induces or promotes apoptosis of proliferating T cells.

6. The method of claim 1, wherein the inhibition of activity comprises inhibiting plasma membrane electron transport in proliferating T cells.

7. The method of claim 1, wherein the proliferating T cells are rapidly or abnormally proliferating T cells.

8. The method of claim 1, wherein the T cells are responder T cells.

9. The method of claim 1, wherein the compound is administered as an immunomodulatory agent, in an immunomodulating effective amount, to a mammal in need thereof.

10. The method of claim 9, wherein the mammal is suffering from, or is susceptible to, a disease or condition associated with abnormal proliferation or stimulation of T cells.

11. The method of claim 9, wherein the immunomodulating effective amount of the compound is less than the therapeutically effective amount where the compound has therapeutic activity against a disease or condition.

12. (canceled)

13. The method of claim 1, wherein the proliferating T cells reside in, or are derived from, an individual suffering from or predisposed to a disease or condition associated with abnormal proliferation or stimulation of T cells.

14. A method of modulating the immune system in a mammal, the method comprising administering to the mammal an immunomodulating effective amount of at least one compound of formula I as described herein or a pharmaceutically acceptable salt or prodrug thereof.

15. The method of claim 14, wherein the compound inhibits the proliferation and/or activity of proliferating T cells.

16. The method of claim 15, wherein the proliferating T cells are abnormally or rapidly proliferating T cells.

17. A method for the treatment or prevention of a disease or condition associated with abnormal proliferation or stimulation of T cells, the method comprising administering to a mammal in need thereof an immunomodulating effective amount of a compound of formula I as described herein or a pharmaceutically acceptable salt or prodrug thereof

18. (canceled)

19. A method for augmenting a treatment regime for a subject suffering from a disease or condition associated with the abnormal proliferation or stimulation of T cells, the method comprising administering to the subject an immunomodulating effective amount of a compound of formula I as described herein or a pharmaceutically acceptable salt or prodrug thereof.
